(19) United States
(12) Patent Application Publication
(10) Pub. No.: US 2011/0200989 A1
(43) Pub. Date: Aug. 18, 2011

(54) SINGLE MOLECULE NUCLEIC ACID SEQUENCING USING MULTIPHOTON FLUORESCENCE EXCITATION

(76) Inventors: Gordon A. Janaway, Hayward, CA (US); Christina E. Inman, San Mateo, CA (US); Joseph Beechem, Eugene, OR (US)

(21) Appl. No.: 13/009,442

(22) Filed: Jan. 19, 2011

Related U.S. Application Data
(60) Provisional application No. 61/296,061, filed on Jan. 19, 2010.

(51) Int. Cl. C12Q 1/68; C12M 1/34; G01N 21/75; G01N 21/63; G01N 21/64

(52) U.S. Cl. 435/6.1; 435/287.2; 436/94; 422/82.09; 422/82.08

(57) ABSTRACT

A system for detection of nucleic acids can include an excitation source configured to transmit excitation energy to a reaction site including a single molecule of nucleic acid reacted with a two-photon absorption moiety. The system also can include an optical system configured to focus the excitation energy transmitted from the excitation source to a focal region containing the reaction site, wherein said excitation energy within the focal region is sufficient to cause two-photon absorption by the two-photon absorption moiety. The system can further include a detector configured to detect emissions generated at the reaction site resulting from two-photon absorption of the excitation energy by the two-photon absorption moiety.

### CROSS-REFERENCE TO RELATED APPLICATIONS

This application claims priority to U.S. Provisional Application No. 61/296,061, filed Jan. 19, 2010, which is incorporated by reference herein in its entirety.

### FIELD

The present teachings relate to fields of nucleic acid (e.g., DNA and all forms of modified DNA, such as methylated DNA, and all forms of RNA, such as microRNA, non-coding RNA, etc.) sequencing, and systems and methods of carrying out the same.

### INTRODUCTION

Single-molecule sequencing has been developed in an attempt to achieve low-cost, high-throughput genomic sequencing. A “sequencing by synthesis” strategy has been used in which polymerase-mediated addition of individual nucleotides (e.g., deoxy-nucleotide triphosphates (dNTP's)) to a template-bound primer is detected. In this approach, each nucleotide to be incorporated to the template-bound primer exhibits a unique optical signature so that the nucleotides act as probes to determine the sequence. Typically this occurs through labeling with four differing fluorescent dyes, so that four-color sequencing can be achieved using a different dye for each of the four nucleotides.

Determination of sequence information from a single nucleic acid molecule in this way requires seemingly contradictory physical conditions, namely, a sufficiently high concentration of unbound dye-labeled nucleotides for efficient polymerization to occur, and a sufficiently low fluorescent background so that individual binding events can be detected. In an attempt to address these requirements, extremely small (e.g., on the order of zeptoliter, 10⁻²¹ L) detection volumes have been used.

In one approach, patterned metallic films are used which contain arrays of holes of about 50 nm in diameter. When illuminated with visible light through a transparent substrate, these wells act as zero-mode waveguides, in which fluorescent excitation is limited to the penetration volume of the near-field evanescent wave. In this way, for each hole, only dye-labeled nucleotides within the volume of the hole are detected. When a single enzyme or primer molecule is confined inside this volume, the addition (incorporation) of individual nucleotides to the growing strand is detected as a step increase in fluorescence which persists for a time longer than the dwell time of unbound (unincorporated) nucleotides. A challenge to this approach, however, is the tight control of surface morphology and surface chemistry required in the patterned waveguide. Non-specific binding of nucleotides and native fluorescence of residual fabrication materials, for example, a photoresist, can lead to a very high fluorescent background. In addition, undesired interactions between the bound polymerase complex and the surface can occur. These considerations can be further complicated by the relatively high aspect ratio of the patterned wells, and the presence of different surface types on the walls and bottom of each well, for example, silica, metal, metal oxide, and the like, all of which can contribute to background fluorescence.

Therefore, a system and method of increasing the signal-to-noise (background) ratio by decreasing the noise and/or increasing the signal would be desirable. Further, a need exists for faster, less expensive, more reliable, single molecule detection and sequencing systems and methods.

### SUMMARY

The present teachings may solve one or more of the above-mentioned problems. Other features and/or advantages may become apparent from the description which follows.

Additional objects and advantages may be set forth in part in the description which follows, and in part will be obvious from the description, or may be learned by practice of the present teachings. Those objects and advantages will be realized and attained by means of the elements and combinations particularly pointed out in the appended claims.

In accordance with at least one exemplary embodiment, the present teachings contemplate a system for detection of nucleic acids that includes an excitation source configured to transmit excitation energy to a reaction site including a single molecule of nucleic acid reacted with a two-photon absorption moiety. The system also can include an optical system configured to focus the excitation energy transmitted from the excitation source to a focal region containing the reaction site, wherein said excitation energy within the focal region is sufficient to cause two-photon absorption by the two-photon absorption moiety. The system can also include a detector configured to detect emissions generated at the reaction site resulting from two-photon absorption of the excitation energy by the two-photon absorption moiety.

In accordance with at least one other exemplary embodiment, the present teachings contemplate a method of detecting nucleic acids that includes transmitting excitation energy to a reaction site at which a single molecule of nucleic acid is immobilized and reacted with a nucleotide labeled with a two-photon absorption dye. The method also includes focusing the transmitted excitation energy to a focal region containing the reaction site, wherein the excitation energy within the focal region is sufficient to cause two-photon absorption by the two-photon absorption dye and detecting emissions generated at the reaction site by the two-photon absorption dye. The method can further include determining a character or sequence of the single molecule of nucleic acid based on the detected emissions.

In accordance with yet at least one other exemplary embodiment, the present teachings contemplate a method of detecting a nucleic acid molecule that includes transmitting excitation energy to a reaction site at which a single molecule of nucleic acid is immobilized and reacted with a polymerase carrying a two-photon absorption moiety, focusing the transmitted excitation energy to a focal region containing the reaction site, wherein the excitation energy within the focal region is sufficient to cause two-photon absorption by the two-photon absorption moiety. The method also includes detecting emissions from an acceptor dye at the reaction site which is excited from fluorescence resonance energy transfer (FRET) from the two-photon absorption moiety. The method can further include determining a character or sequence of the nucleic acid molecule based on the detected emissions.

It is to be understood that both the foregoing general description and the following detailed description are exemplary and explanatory only and are not restrictive of the present teachings or claims.

### BRIEF DESCRIPTION OF THE DRAWINGS

The accompanying drawings, which are incorporated in and constitute a part of this specification, illustrate various exemplary embodiments of the present teachings and together with the description, serve to explain certain principles. In the drawings:

FIG. 1A depicts a one-photon absorption and fluorescence scheme;

FIG. 1B depicts a two-photon absorption and fluorescence scheme according to various exemplary embodiments of the present teachings;

FIG. 2 shows the area of a confined detection volume that can be monitored to detect two-photon absorption according to various exemplary embodiments of the present teachings;

FIG. 3 is a side view of a system according to various exemplary embodiments of the present teachings, wherein a laser excitation beam is used to cause two-photon absorption of a tethered nucleic acid building complex bound to a transparent substrate; and

FIG. 4 is a diagram of a system according to various exemplary embodiments of the present teachings, wherein an excitation beam is provided to a plurality of reaction sites to cause two-photon absorption at the reaction sites.

### DETAILED DESCRIPTION

Reference will now be made in detail to various exemplary embodiments, some of which are illustrated in the accompanying drawings. Wherever possible, the same reference numbers will be used throughout the drawings to refer to the same or like parts.

To facilitate an understanding of the present teachings, the following definitions are provided. It is to be understood that, in general, terms not otherwise defined are to be given their ordinary meanings or meanings as generally accepted in the art.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. Thus, for example, reference to “a sample” can include two or more different samples. As used herein, the term “include” and its grammatical variants are intended to be non-limiting, such that recitation of items in a list is not to the exclusion of other like items that can be substituted or added to the listed items.

Various exemplary embodiments of the present teachings contemplate a single nucleic acid molecule sequencing system and method that provide for high throughput, low copy-number sequencing applications. According to various exemplary embodiments, volume confinement of fluorescent detection is provided through the use of multi-photon (including, for example, two-photon) absorption. Such multi-photon absorption can obviate the need for patterned substrates utilizing sub-wavelength scale holes or other reaction chambers. Many fluorescent dyes that exhibit large one-photon absorption can also be used for multi-photon (e.g., two-photon) absorption, according to various exemplary embodiments of the present teachings. In addition, through the use of specially-designed chromophores with large two-photon cross-sections, exceptionally low background signal can be achieved. Suitable exemplary two-photon absorption dyes and design of the same can be found, for example, in M. Pawlicki et al., “Two-photon absorption and the design of two-photon dyes,” Agnew. Chem. Int. Ed., Vol. 48, pp. 3244-3266 (2009).

According to various exemplary embodiments of the present teachings, a method and system for single-molecule nucleic acid sequencing through the use of multi-photon (e.g., two-photon) absorption optically probes nucleotide incorporation events in small volumes of concentrated solutions. In some embodiments, a sequencing-by-synthesis approach can be used that involves detecting individual nucleotides using two-photon absorption as the individual nucleotides are incorporated into a growing nucleic acid strand. In some embodiments, to provide a continuous read, a polymerase complex can be bound to a transparent substrate either through covalent attachment of a polymerase enzyme, primer nucleic acid, or template nucleic acid. In contrast to a zero-mode waveguide approach, the present teachings do not require an intricate patterning of a transparent substrate or the introduction and confinement of a single molecule of nucleic acid within an extremely small volume reaction chamber. Instead, the present teachings permit utilization of a bulk flow of sample, for example, in a flow cell or other volume with single molecule templates bound to substantially planar surface.

In various exemplary embodiments, a detection system is provided that comprises an excitation source configured to transmit excitation energy at a first wavelength range that includes a first wavelength to a reaction site including a nucleic acid molecule reacted with a two-photon absorption dye to generate an emission signal at the reaction site. An optical system is provided that is configured to focus the excitation energy transmitted from the excitation source to a focal volume in which the reaction site is located to cause two-photon absorption of the excitation energy of the first wavelength within the first wavelength range by the two-photon absorption dye. The two-photon dye may include, but is not limited to, for example, a chromophore or a rhodamine dye, quantum dot nanocrystal, squaraines, conjugated porphyrins, and aromatic expanded porphyrin analogues. The system can further comprise a detector configured to detect the emission signal from the reaction site, emitted from within the focal area.

In some embodiments, the excitation source comprises a laser source, which may be a pulsed laser source or a constant wavelength laser source. When a pulsed laser source is used, in various exemplary embodiments, the power may range from about 10 milliwatts to about 100 milliwatts, for example, about 50 milliwatts. For a constant wavelength laser source, in various exemplary embodiments, the power may range from about 0.1 Watts to about 10 Watts, for example, about 1 Watt.

In some embodiments, the optical system is configured for confocal detection, and comprises a beam splitter, a confocal lens, a combination thereof, and the like, these components being well-known to those of ordinary skill in the art. In some embodiments, the optical detection path is not coincident (confocal) with the excitation path. In some embodiments, the detector is provided with a detector filter, such as, for example, a spectral filter, that filters out wavelengths of light including wavelengths within the excitation wavelength range and longer. In some embodiments, the detection system further comprises a flow cell, wherein the reaction site is within the flow cell. The flow cell can comprise a transparent window or coverslip in some embodiments.

According to various exemplary embodiments, methods and systems of detecting a nucleic acid molecule, for example, DNA, is provided. The method can comprise immobilizing a single nucleic acid molecule at a reaction site, reacting the nucleic acid molecule with a two-photon absorption molecule, such as, for example, a two-photon absorption dye, and exciting the two-photon absorption molecule with energy (e.g., radiation), after the reaction with the nucleic acid molecule, and cause the two-photon absorption molecule to undergo two-photon absorption. The method can further comprise detecting emission from the two-photon absorption molecule that results from the two-photon absorption, and determining a character or sequence of the nucleic acid molecule based on the detected signal. In some embodiments, determining a character or sequence of the nucleic acid molecule can comprise sequencing the nucleic acid molecule. In some embodiments, a change in color and/or intensity can be detected when a nucleotide is covalently attached to a template. In some embodiments, a change in the persistence length of fluorescence upon incorporation is detected, for example, if a nucleotide is not incorporated the fluorescent signal can fall rapidly upon the nucleotide diffusing away from the focal site, for example, out of the focal volume of the containing the reaction site. If the nucleotide is incorporated, fluorescence should last much longer due to the incorporated nucleotide being held within the focal volume of the excitation energy.

In various exemplary embodiments, the excitation energy is transmitted at a first wavelength that is outside of the absorption band of the dyes used with the dye-labeled nucleotides, for example, the first wavelength may be higher than the wavelengths of the absorption band of the dyes. The excitation energy may be modified, for example, via focusing, from the excitation source to the reaction site so as to increase its intensity upon reaching the reaction site sufficiently to achieve two-photon absorption of the excitation energy reaching the reaction site. In various exemplary embodiments, the excitation energy outside of a focal volume encompassing the reaction site will not have an intensity sufficient to achieve two-photon absorption. Further, when the wavelength of the excitation energy is not within the absorption band of the dyes on the dye-labeled nucleotides, it will not be absorbed by dyes outside the focal volume as it is transmitted from the source to the reaction site. In this way, various exemplary embodiments achieve a significant reduction in fluorescence emissions from unincorporated dyes present outside the focal volume during an incorporation event.

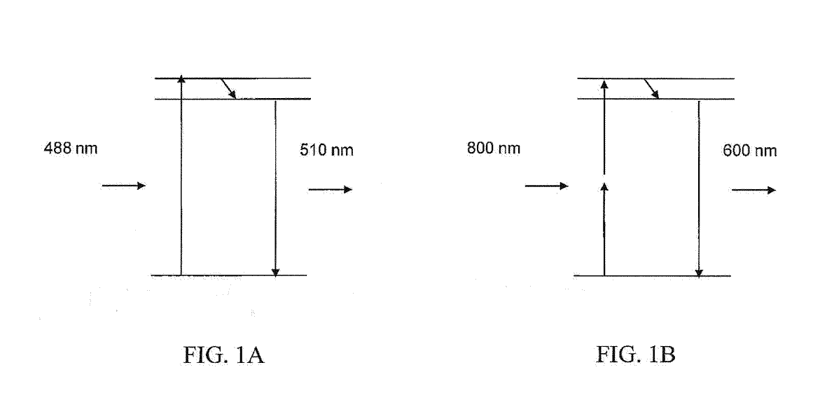

With reference now to the drawings, one-photon excitation of a dye molecule is schematically illustrated in FIG. 1A. The excitation is followed by internal conversion and subsequent fluorescent radiation, at a wavelength longer than that of the absorbed light. For example, with an exemplary dye molecule, an excitation beam having a wavelength of 488 nm can result in an emission at a wavelength of 510 nm. In accordance with Beer's Law, the probability of one-photon absorption is directly proportional to the intensity of the excitation beam.

According to various exemplary embodiments of the present teachings, a system is provided to enable two-photon absorption of a dye molecule. In some embodiments, sufficiently intense excitation energy (e.g., radiation) is used to cause two-photon absorption to occur. Two photons, each equal to half the energy of the observed transition, are absorbed nearly simultaneously by a dye molecule within a focal volume to which the excitation energy is directed and focused. Following absorption, the processes of internal conversion and radiative relaxation result in the emission of fluorescence that can be shorter in wavelength than the excitation source. For example, excitation at an 800 nm wavelength can result in emission at a 600 nm wavelength, as is schematically illustrated in FIG. 1B. The probability of two-photon absorption is proportional to the square of the excitation intensity, and therefore the probability of achieving two-photon absorption also falls off dramatically as the excitation energy intensity decreases.

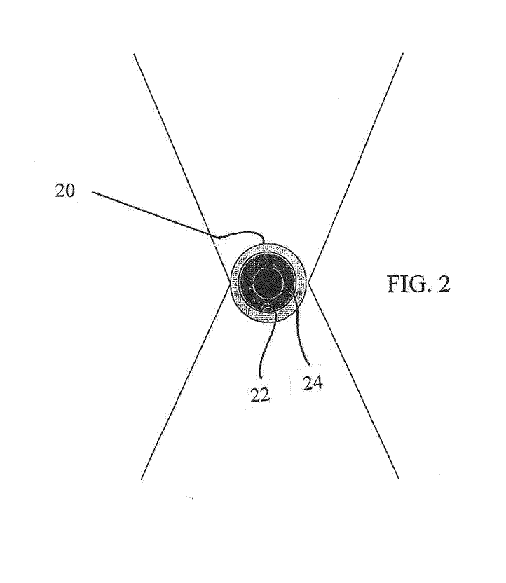

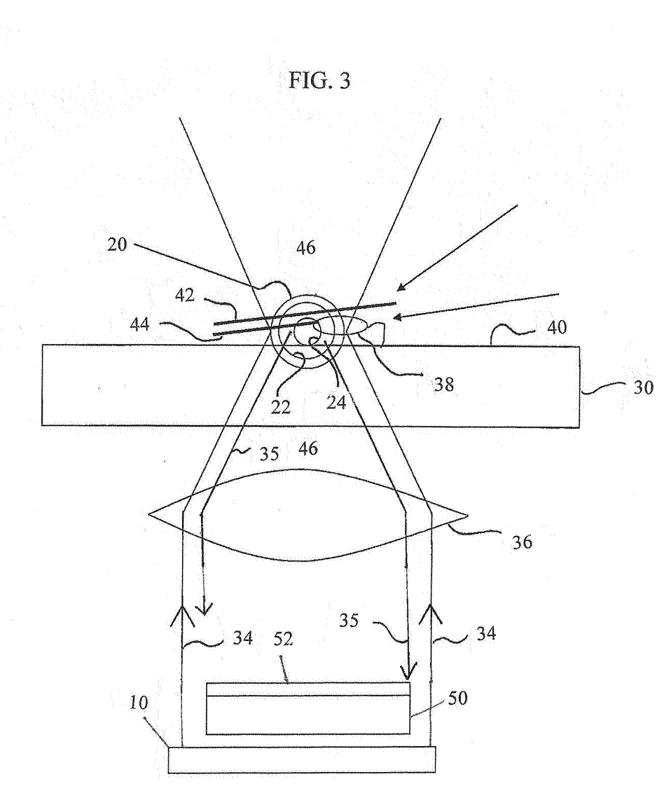

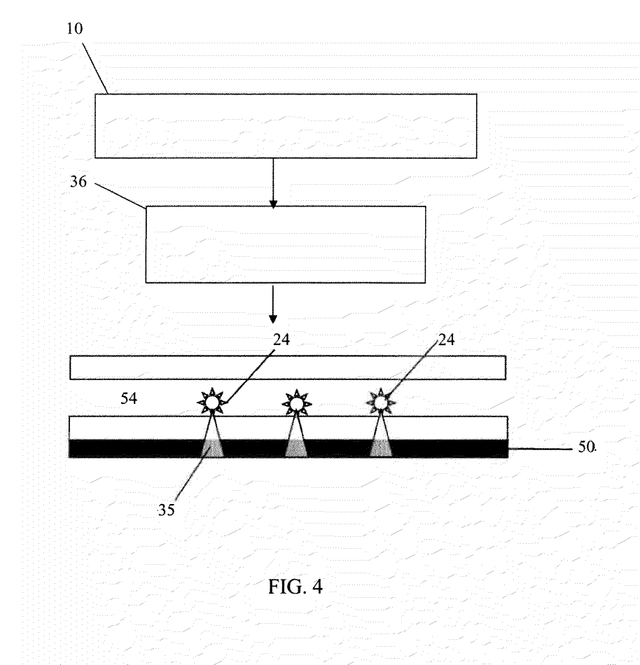

In a focused laser beam, the light intensity decreases along the optical axis roughly as z² away from the focal point. Since the probability of two-photon absorption is proportional to the square of intensity (I²), the probability of two-photon absorption decreases as z⁴ from the focal point along the optical axis. According to various exemplary embodiments, essentially no two-photon absorption occurs outside of a small focal volume defined at or within the focal region of the laser. As can be seen in the exemplary depiction in FIG. 2, light from an excitation source 10 (see FIGS. 3 and 4) can be focused to form a focal region 20 having a diameter, and a small, confined detection volume 22 can be located wholly within focal region 20. A reaction site 24 at which dye-nucleotide incorporation into a single molecule of nucleic acid can be substantially centered and wholly within detection volume 22. It should be noted that in the two-dimensional illustration of FIG. 2, the volumes 20, 22, and 24 are depicted as generally circular areas. In the embodiment shown in FIG. 2, detection volume 22 has a diameter that is larger than the diameter of reaction site 24 and smaller than the diameter of focal region 20. In some embodiments, by adjusting the intensity of excitation, the detection volume 22 has a diameter that is from about 5% to about 90% of the diameter of focal region 20, for example, from about 10% to about 75%, from about 15% to about 50%, from about 20% to about 40%, or about 30%, of the diameter of the focal region. In other words, the detection volume 22 is a focal volume within which the intensity is high enough to achieve two-photon absorption by two-photon absorption dyes located within that volume. In some cases, this focal volume 22 may be coincident with the focal region 20. In various exemplary embodiments, the detection or focal volume 22 sufficient to achieve two-photon absorption ranges from about 0.1 femtoliters to about 10 femtoliters, for example, the volume is about 1 femtoliter.

According to various exemplary embodiments, two-photon absorption can be used in a system and method for single-molecule detection. In some embodiments, a method of single-molecule nucleic acid sequencing is provided through the use of two-photon or multi-photon absorption to optically probe nucleotide additions in small volumes of concentrated solutions. Although two-photon absorption is primarily exemplified herein, it is to be understood that multi-photon absorption can be useful as well, according to the present teachings. The method can comprise a sequencing-by-synthesis approach, wherein individual nucleotides can be detected using two-photon absorption as they are incorporated into a growing single nucleic acid molecule strand.

Various exemplary embodiments of the present teachings can be more fully understood with reference to FIGS. 3 and 4. FIG. 4 shows an overview system diagram of an exemplary embodiment of the present teachings including an excitation source 10, which may be a laser source, providing excitation energy to an objective lens 36. The excitation energy may be provided to a plurality of reaction sites 24 located within a flow cell 54. The objective lens 36 focuses the excitation energy provided from the excitation source 10 to respective focal regions containing the reaction sites 24. As will be discussed below, two photons of excitation energy are substantially simultaneously absorbed by two-absorption dyes present at the reaction sites, including a dye incorporated at the reaction site 24. Following the two-photon absorption, emission beams 35 are generated at the reaction sites 24 resulting from two-photon absorption of the excitation energy by the two-photon absorption dyes. The detector 50 detects the emission beams 35 generated at the reaction site resulting from the two-photon absorption of the excitation energy by the two-photon absorption dye. While FIG. 4 shows a flow cell 54 provided with multiple reaction sites 24 as an example of a sample chamber, any of a variety of sample chambers may be provided which are able to receive a single molecule of nucleic acid reacted with a two-photon absorption moiety and which allow for two-photon absorption at a reaction site. In addition, while the excitation source 10 is located above the flow cell 54 and the detector 50 is located below the flow cell 54 in FIG. 4, such an arrangement is merely exemplary, and the excitation source 10 and the detector 50 may be located at the same side of the flow cell 54 (see FIG. 3, for example).

The following description of FIG. 3 and the methodology is explained in terms of a single reaction site for purposes of simplification, and it may be recognized by those of ordinary skill in the art that multiple reaction sites, as shown and described in FIG. 4, for example, may be provided. FIG. 3 shows a transparent substrate 30, a reaction site 24 on the transparent substrate 30, a laser beam path 34, and an objective lens 36 along laser beam path 34. The reaction site 24 includes a single molecule of a template nucleic acid strand for which detection and/or sequencing is desired, and upon an incorporation event, a nucleotide labeled with a two-photon absorption dye that reacts (covalently binds) to the template molecule. The laser beam path 34 is defined from excitation source 10 to the reaction site 24 that includes the bound nucleic acid molecule that is reacted with the dye-labeled incorporated nucleotide labeled with a two-photon absorption dye. The excitation source 10, which in exemplary embodiments can be a pulsed laser or a constant wavelength laser as described above, is configured to transmit energy at a wavelength outside (e.g., higher than) the one-photon wavelength range of absorption of the dyes labeling the various nucleotides that are introduced.

Laser beam path 34 is incident on the reaction site 24 within the focal region 20. A confined detection volume or focal volume 22 is wholly within focal region 20. In the embodiment shown in FIG. 3, detection volume 22 has a diameter that is larger than the diameter of reaction site 24 and smaller than the diameter of focal region 20. A tethered polymerase enzyme complex 38 can be tethered to a top surface 40 of transparent substrate 30 along with the single molecule of a template nucleic acid sequence 42. The template nucleic acid molecule 42 can be reacted with individual dye-labeled nucleotides introduced above the surface of the substrate 30 (e.g., in a flow cell defined by the substrate 30) to form a growing complementary strand 44 that is complementary to template molecule 42. The strand 44 grows as dye-labeled nucleotides are incorporated one at a time. To ensure a continuous read, polymerase enzyme complex 38 is bound to transparent substrate 30 either through a covalent attachment of a polymerase enzyme or through a covalent attachment of the primer nucleic acid, such as DNA.

The beams transmitted from the laser source 10 are focused by the objective lens 36 to the focal region 20. The focusing of the beams 34 to the small focal region 20 increases the intensity of the transmitted energy at least within the detection or focal volume 22 sufficiently to permit two photons of excitation energy to be substantially simultaneously absorbed by two-absorption dyes present within the focal volume 22, including a dye incorporated at the reaction site 24. In particular, if the two-photon absorption dye absorbs two photons simultaneously, the dye will be excited and emit a signal. As two photons are absorbed during the excitation of the two-photon absorption dye, the probability for emission from the two-photon absorption dye increases quadratically with the intensity of the excitation energy. Thus, the laser beam 34 is focused to the focal volume 22 that includes two-photon absorption dyes, including a dye on a dye-labeled incorporated nucleotide, to cause two-photon absorption of the excitation energy by the two-photon absorption dye within the focal volume 22.

Fluorescent emissions from the two-photon absorption event are emitted as emission beams 35 that can be directed along the same or substantially the same optical pathway taken in an opposite direction by excitation beam 34.

Because the wavelength of the excitation energy from the laser source 10 is at a longer wavelength than the absorption band of the dyes labeling the dye-labeled nucleotides, dye-labeled nucleotides that are located outside of the focal volume 22 will not be excited by transmitted energy from the laser source 10, even when those nucleotides are in the laser beam path (e.g., the region inside the cones 46 and outside the focal volume 22). This is because one-photon absorption is not possible in light of the excitation wavelength being outside the absorption band of the dyes and the intensity of the excitation energy in those regions being insufficient (too low) to enable two-photon absorption by the dyes. Thus, by sufficient focusing of the laser beam 34, two-photon absorption can be limited to the small focal volume 22 that contains the absorption dye that has been reacted with the nucleic acid molecule at the reaction site 24. Hence, the large sample volume containing unincorporated dye-labeled nucleotides outside the focal volume 22 will not result in significant direct excitation of unincorporated absorption dyes (e.g., the dye-labeled nucleotides that are not reacted with the nucleic acid molecule at the reaction site 24).

In addition, using the system and method of FIG. 3, background noise can be further reduced and/or distinguished from the signal of interest since unincorporated dye-labeled nucleotides that are within the focal volume 22 that are excited will have the ability to diffuse out of the focal volume 22. In this way, the emission signal that is detected from those emitting dyes that absorbed two-photons will be transient and short-lived compared to the emission signal from the dye labeling the incorporated nucleotide at the reaction site 24.

The exemplary system of FIG. 3 also includes a detector 50 that is configured to detect emissions emitted as a result of the two-photon absorption, including the emission from the dye-labeled nucleotide incorporated to grow the complementary strand 42 at the reaction site 24. The exemplary system of FIG. 3 may also include a detector filter 52 that filters out wavelengths of light including wavelengths within a first wavelength range and longer.

In one exemplary embodiment, discussed in more detail below, a fluorescence resonance energy transfer (FRET) dye system may be used in which an emission signal may be emitted from an acceptor dye that labels an incorporated nucleotide and is in close proximity to a donor two-photon absorption dye reacted with the nucleic acid molecule and located at the reaction site 24, the detected emissions being generated by the acceptor dye after excitation and two-photon absorption by the donor dye with fluorescence resonant energy transfer and excitation of the acceptor dye.

In contrast to the zero-mode waveguide approach, the system of FIG. 3 does not require, but also does not preclude, intricate patterning of transparent substrate 30 and/or confinement of the single molecule of template nucleic acid 44, polymerase 38, and complementary strand 42 within a very small-volume reaction chamber (hole) defining the zero-mode waveguide. Top surface 40 can be an inner surface of a flow cell, for example, a transparent coverslip that defines an inner surface of the flow cell.

According to various exemplary embodiments, a primer sequence, nucleic acid template, or polymerase molecule can be tethered to a glass surface through a covalent linkage, for example, an amide bond created by reacting an NHS ester-coated surface with an amine functionalized template or enzyme, a siloxide linkage, a sulfide linkage, a streptavidin-biotin linkage, or the like. Surface coverage of the bound primer can be sufficiently low such that two primers are far enough apart (in space) to be observed as distinct objects by the imaging system. In some cases, surface coverage of the bound primer can be sufficiently low such that on average there are from about 0.2 to about 0.6, or about 0.4, primer per focal volume. A buffered solution of polymerase enzyme and template, for example, also containing Mg2+ and dNTPs, can be added. A laser source can be used to irradiate the solution near the surface. In some embodiments, optics, for example, four-color confocal optics, can be used to detect up-converted fluorescence resulting from two-photon absorption near the laser focal volume.

In some embodiments, a background level of fluorescent signal can be observed from the enzyme, primer, and template. Superimposed on this background, short bursts of fluorescence can be observed from diffusion of individual nucleotides through the focal volume. When a nucleotide is added to the growing complementary strand, a persistent increase in fluorescence can be observed until photobleaching of the fluorophore occurs.

In some embodiments, the native two-photon absorption cross-sections and emission quantum efficiencies of each nucleotide are sufficiently large for detection above background. Alternatively, each of the four nucleotides (dNTPs) could be labeled with a different two-photon absorption fluorophore. With properly designed labels, very low background signals with low laser power (e.g., on the order of 1 milliwatt for pulsed laser sources) can be achieved. While a variety of dyes can be used to label the nucleotide probes in accordance with the present teachings, including, for example, FAM, VIC, fluorescein dyes, rhodamine dyes, and the like, in some embodiments the dyes described in the article of Cumpston et al., Two-photon polymerization initiators for three-dimensional optical data storage and microfabrication, Nature 398, 6722, 51 (1999), can be used, and such reference is incorporated herein in its entirety by reference.

According to various exemplary embodiments of the present teachings, a polymerase enzyme can be tethered to a substrate surface, instead of a primer. In some embodiments, irradiation can be directed from above the sequencing solution, for example, such that a transparent substrate is not required. In some embodiments, the substrate surface can be coated to diminish non-specific binding and/or to provide attachment points for the attachment of template, primer, and/or polymerase.

In yet other embodiments of the present teachings, two-photon absorption has been shown to increase dramatically for chromophores in solution near fractal metal films, causing a so-called “antenna effect”. In some embodiments, a fractal metal film is patterned on a glass surface prior to attachment of a tethered enzyme or primer. When probed near the fractal metal, two-photon absorption can be enhanced for all chromophores in solution (background and signal), and a lower laser power can be used compared to when no chromophores are utilized.

Although most organic molecules undergo two-photon absorption under sufficient energy intensities, according to various embodiments of the present teachings, specialized chromophores with large coefficients of two-photon absorption, designed for imaging and lithography applications, can be used. Resolutions of two-photon absorption-lithography can be used to produce features as small as 100 nm, as small as 80 nm, as small as 60 nm, as small as 40 nm, or as small as 30 nm, or from about 20 nm to about 30 nm. Some exemplary imaging systems and optics that can be used include those described, for example, in the article of Miller et al., Two-Photon Imaging of Lymphocyte Motility and Antigen Response in Intact Lymph Node, Science 296, 5574, 1869-1873 (2002), which is incorporated herein in its entirety by reference.

According to various embodiments, single-molecule detection using two-photon absorption can be achieved in bulk solution. The method can comprise binding a primer, template, or polymerase to a fixed planar surface. In some embodiments, binding or attachment can be to a sharp point, such as an AFM tip. Binding can be sufficient to effect immobilization of the primer or enzyme. In some embodiments, an increase in two-photon absorption confinement and a reduction in focal volume results due to the enhanced electric field in the proximity of the curved metal surface.

In some embodiments, a template can be attached to a lipid bilayer such that the position of the nucleic acid strand or polymerase is controlled with fluid dynamics. In some embodiments, attaching a nucleic acid template to a lipid bilayer can be used to manipulate the nucleic acid template in a flow cell.

In some embodiments, a low-density gel matrix can be used to immobilize a polymerase/primer/template complex. Immobilization can occur, for example, through covalent attachment, physical entrapment, or the like. The matrix can allow diffusion of dye-labeled nucleotides. In some embodiments, a high viscosity solution can be used to improve length-of-read by reducing diffusion of the polymerase complex through the gel.

According to various embodiments, systems and methods for parallel sequencing are provided which use two-photon absorption. In some embodiments, two-photon fabrication using far-field masks and utilizing rapid two-photon absorption over large areas enable massively parallel single-molecule sequencing. In some embodiments, holographic, two-beam excitation is provided over large areas, for example, as described in Kirkpatrick et al., Holographic recording using two-photon-induced photopolymerization, Journal Applied Physics A: Materials Science & Processing; Issue Volume 69, Number 4, October, 1999, which is incorporated herein in its entirety by reference.

According to various embodiments, a nucleic acid detection system is provided that comprises a two-photon absorption and fluorescence system as described herein, and a detector. The detector can be configured to receive a fluorescent signal from a reaction site. Dyes can be used that can be excited by the two-photon absorption and fluorescence system. In some embodiments, a set of dyes is used, for example, a set of four different dyes. In some embodiments, the detector can comprise one or more charge-coupled device (CCD) cameras, or complementary metal oxide semiconductor (CMOS) cameras. In some embodiments, the system can further comprise a reaction site and a direct or indirect binding or reaction of a quantum dot, a nucleic acid molecule, a polymerase, and a dye-labeled nucleotide at the reaction site. The reaction site can be on a thin glass substrate or film, for example, on a silica plate or film. Imaging can be done using a scanning system or using, for example, a holographic optical component useful for dividing a laser beam into a plurality of rays of radiation which can be directed simultaneously at a respective plurality of reaction sites. The optical pathway can be the same, at least for a portion of its length, for both the excitation beam and the emission beam. Beam splitters, diffraction gratings, prisms, filters, and the like can be used in the optical pathway(s), for example, as are common in real-time polymerase chain reaction (PCR) detection instruments and generally known to those of ordinary skill in the art.

In some embodiments, the method can comprise exciting the two-photon absorption molecule with radiation of a first wavelength before, during, and/or after the reaction with the single molecule of template nucleic acid.

According to various embodiments, a method of detecting a nucleic acid molecule is provided, and comprises detecting interactions of a dye-labeled nucleotide molecule that is labeled with a two-photon absorption dye. In some embodiments, the detecting can comprise forming a detected signal, and the method can further comprise determining a base or sequence of the nucleic acid molecule based on the detected signal. In some embodiments, the method can comprise sequencing the nucleic acid molecule.

According to various embodiments, fluorescence resonance energy transfer (FRET) dye systems, which may use quantum dots or other long lifetime fluorescent dyes as donor species, for example, may be used in conjunction with a two-photon absorption and fluorescence system as described herein. Two-photon excitation can be particularly useful when performing sequencing reactions using FRET-based dye systems and signal generation. FRET-based signal generation utilizes at least two distinct fluorescent emitting species—the “donor” and the “acceptor.” Excitation energy is absorbed by the donor species, and if the emission wavelength of the donor overlaps with the absorption spectrum of the acceptor, then the excited state of the donor can be resonantly transferred to the acceptor.

In a single molecule sequencing approach, the donor emitting species is the nucleic acid polymerase and the acceptor emitting species that provides the nucleic acid sequence-dependent signal is the incorporated (to the nucleic acid polymerase) dye-labeled nucleotide. Using one-photon absorption FRET, the excitation-wavelength is at shorter wavelengths that are more typically within or lower than the absorption band of the acceptor. Hence, the fluorescence emission signals that are detected can be complicated by excitation that goes directly into the absorption band of the acceptors, since the excitation at shorter wavelengths will typically be capable of being absorbed by the acceptors. This “directly-excited” acceptor population will generate fluorescence acceptor emissions that are spectrally identical to the acceptor fluorescence emission obtained through the FRET pathway. Thus, the emission of the directly-excited acceptor can generate a potentially large sequence-independent background signal, making detection of the sequence-dependent FRET-based acceptor emissions (i.e., emissions from an incorporated nucleotide labeled with an acceptor dye) difficult. To ensure rapid kinetics and efficiency of single molecule sequencing reactions, particularly in FRET-based systems, relatively high concentrations (e.g., hundreds of nanomolars to a few micromolar concentrations) of acceptor dye-labeled nucleotides are used. This high concentration of acceptor dyes further exacerbates the problem of background fluorescence by increasing the directly-excited acceptor dye background signal relative to the low concentration (nanomolars) of the donor-emitting species. For a further explanation of the use of FRET-based dye systems and methods useful for single nucleic acid molecule detection and/or sequencing, reference is made to International Publication No. WO/2010/111674, entitled “Methods and Apparatus for Single Molecule Sequencing Using Energy Transfer Detection,” which is incorporated by reference herein in its entirety.

In order to aid in eliminating this large background signal associated with FRET-based dye systems for single molecule nucleic acid detection, two-photon excitation and absorption in accordance with the present teachings can be utilized in conjunction with FRET-based dye systems. For example, the wavelength of the energy (irradiation) that impinges on the sample within the focal volume is at a longer wavelength (lower energy) than the absorption-band of the acceptor species (i.e., the acceptor dyes labeling the nucleotide probes). In this manner, there will be essentially no one-photon direct excitation of the acceptor dyes within this volume or outside of this volume where a large concentration of unincorporated acceptor dyes are present. By suitable focusing of the excitation light, the two-photon absorption can be limited to the small sample volume in the focal volume (e.g., the focal volume 22 shown in the embodiment of FIG. 3) that contains the donor-species of interest (e.g., the donor, which is a two-photon absorption species, tethered to the polymerase that is located at reaction site 24). Therefore, the relatively large concentration of FRET acceptor dyes labeling unincorporated nucleotides in the sample volume outside of the focal volume and within the cones 46 will not be directly excited by the long wavelength, low intensity irradiation energy transmitted from the excitation source 10. Instead, due to the focusing of the excitation energy within the focal volume and its consequent increase in intensity, two-photon absorption by the donor species can occur to excite the donor species. Subsequently, as a result of FRET, the acceptor species on the incorporated labeled nucleotide at the reaction site and in sufficiently close proximity to the excited donor will become excited and generate emissions due to the FRET excitation received from the donor. Those FRET emissions can then be detected to determine the character or sequence of the immobilized nucleic acid molecule at the reaction site.

As a specific example, consider a donor-labeled nucleic acid polymerase with a single-photon extinction coefficient of donor absorbance (eda) and a single-photon extinction coefficient of acceptor (eaa). The following apply:

Total Absorbance of sample (one-photon)=[donor]×eda[one-photon wavelength]+[acceptor]×eaa[one-photon] Equation 1:

Equation 1 is applicable in the irradiated volumes (e.g., defined within the cones 46 in the exemplary embodiment of FIG. 3) above and below the focal volume of the excitation as well as inside the focal volume (e.g., defined by 20 in the exemplary embodiment of FIG. 3).

For two-photon excitation conditions, Equations 2a and 2b below are illustrative, where a two-photon extinction coefficient of donor absorbance is represented as “tpeda” and a two-photon extinction coefficient of acceptor is represented as “tpeaa”.

Total Absorbance of sample (two-photon)=[donor]×tpeda[two-photon wavelength]+[acceptor]×tpeaa[two-photon wavelength] Equation 2a (in the two-photon excitation volume element):

Total Absorbance of sample (one-photon)=[donor]×eda[two-photon wavelength]+[acceptor]×eaa[two-photon wavelength] Equation 2b (outside of the two-photon excitation volume element):

Since the two-photon excitation wavelength is well beyond (e.g., higher than) the single-photon absorption band of both the donor and the acceptor, the out-of-focal volume signal in these systems is greatly diminished (eda and eaa are essentially zero at the two photon wavelength). While for non-FRET based nucleic acid detection/sequencing systems two-photon excitation is very useful, for FRET-based systems, especially when the concentration of the acceptor species is often required to be in vast excess of the donor species, two-photon excitation approaches provide for the benefit of effectively removing the otherwise relatively large background noise that would be generated outside of the small focal volume in which the reaction site at which the template nucleic acid molecule, donor-labeled polymerase, and acceptor-labeled incorporated nucleotide are located.

Some examples of suitable donor and acceptor moieties and their associated single- and two-photon absorption wavelengths that can be used with a two-photon FRET-based dye system for single molecule nucleic acid detection and/or sequencing in accordance with the present teachings are presented in Table 1 below:

TABLE 1
Examples of potential two-photon FRET-based sequencing systems

| Donor molecules for- | Acceptor dyes for | Single-photon | Two-photon |
|---|---|---|---|
| labeling nucleic acid | labeling nucleotides- | excitation | excitation |
| polymerase | | wavelength | wavelength |
| | | (nm) | (nm) |
| Alexa Fluor 555 | Alexa Fluor 647 | 532 | 1064 |
| CdSe quantum dot | Alexa Fluor 647 | 405 | 810 |
| sequencing | | | |
| nanocrystal | | | |
| Alexa Fluor 488 | Alexa Fluor 555 | 488 | 976 |

According to various exemplary embodiments, a detection scheme that implements a fluorescence lifetime imaging (FLIM) technique may be used in conjunction with the fluorescence resonance energy transfer (FRET), two-photon absorption dye systems described above to further improve (e.g., increase) the signal-to-noise ratio. Since direct excitation via two-photon absorption by the acceptor dyes within the focal volume may occur, resulting in emissions from the acceptor dyes that are not a result of the emissions generated due to the FRET excitation by the donor, it may be desirable to filter out detection of such emissions resulting from direct excitation and two-photon absorption of unincorporated acceptor dyes within the focal volume. In an exemplary embodiment, to filter such emissions, the detector may be able to distinguish direct emissions from those unincorporated acceptors from emissions from an incorporated acceptor generated as a result of the FRET excitation by the donor. In at least one exemplary embodiment, this filtering can be achieved, for example, via a timed detection or gating scheme that relies on fluorescence lifetime imaging (FLIM) principles. For a further explanation of how fluorescence lifetime imaging and time-gated detection can distinguish the FRET-based emissions from the emissions resulting from direct excitation of acceptors (e.g., via two-photon absorption in the present case), as well as for a further description of FRET-based dye systems, for single nucleic acid molecule detection and/or sequencing, reference is made to U.S. patent application Ser. No. 12/980,817, entitled “Single Molecule Detection and Sequencing Using Fluorescence Lifetime Imaging,” filed on Dec. 29, 2010, the entire disclosure of which is incorporated herein by reference.

In some embodiments, electron multiplying, electron bombardment, or low noise CMOS detectors can be utilized as the detector 50. In some embodiments, the method can comprise reacting a template nucleic acid molecule with a two-photon absorption molecule or reacting the nucleic acid molecule with a second molecule that comprises a two-photon absorption moiety.

Other two-photon absorption systems and methods that can be used according to the present teachings include those described, for example, in U.S. Pat. No. 7,052,847 to Webb et al, The article of Miller et al., Two-Photon Imaging of Lymphocyte Motility and Antigen Response in Intact Lymph Node, Science 296, 5574, 1869-1873 (2002), the article of Maruo et al., Three-dimensional microfabrication with two-photon-absorbed photopolymerization, Opt. Lett. 22, 132-134 (1997), the article of Mertz et al., Single-molecule detection by two-photon-excited fluorescence, Opt. Lett. 20, 2532 (1995), the article of Cumpston et al., Two-photon polymerization initiators for three-dimensional optical data storage and microfabrication, Nature 398, 6722, 51 (1999), the article of Juodkazis et al., Two-photon lithography of nanorods in SU-8 photoresist, Nanotechnology 16 846-849 (2005), the article of Wenseleers et al., Five orders-of-magnitude enhancement of two-photon absorption for dyes on silver nanoparticle fractal clusters, J. Phys. Chem. B, 106, 6853-6863 (2002), the article of Jeon et al, Fabricating three dimensional nanostructures using two photon lithography in a single exposure step, Optics Express, 14 (6): 2300 (2006), and the article of Jeon et al., Fabricating three dimensional nanostructures using two photon lithography in a single exposure step, Optics Express, 14 (6): 2300 (2006), each of which is incorporated herein in its entirety by reference.

Other excitation sources, detectors, electronics, processors, components, methods, and the like, that can be used according to the present teachings include those described, for example, in U.S. Published Patent Application No. US 2009/0146076 A1 to Chiou et al., the article of Poher et al., Video Rate Fluorescence Lifetime Imaging and Structured Illumination Using a Blue LED, Imaging Sciences Centre, Photonics Group, Department of Physics, Imperial College London, and in the publication from crackerbio entitled Description of our technology, cracker, from cracker[@] ITRI, www.crackerbio.com, each of which is incorporated herein in its entirety by reference.

Further modifications and alternative embodiments will be apparent to those skilled in the art in view of the disclosure herein. For example, the systems and the method may include additional components or steps that were omitted from the diagrams for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

Those having skill in the art would recognize that the various exemplary embodiments described herein may be modified to perform a variety of assays, and although some specific examples for which the systems and methods may be well-suited are disclosed, such examples are nonlimiting and exemplary only. By way of example, various excitation and absorption wavelengths are disclosed, which may be suitable for use in conjunction with various exemplary embodiments. However, based on the present teachings, those having ordinary skill in the art would understand how to select such parameters depending for example, on the dyes used and/or other factors, in order to carry out the methods and systems taught herein.

It is to be understood that the particular examples and embodiments set forth herein are nonlimiting, and modifications to structure, dimensions, materials, and methodologies may be made without departing from the scope of the present teachings.

Other embodiments will be apparent to those skilled in the art from consideration of the specification and practice of the present teachings disclosed herein. It is intended that the specification and examples be considered as exemplary only, with a scope being of a breadth indicated by the claims, including their full scope of equivalents.

What is claimed:

1. A system for detection of nucleic acids, the system comprising:
an excitation source configured to transmit excitation energy to a reaction site including a single molecule of nucleic acid reacted with a two-photon absorption moiety;
an optical system configured to focus the excitation energy transmitted from the excitation source to a focal region containing the reaction site, wherein said excitation energy within the focal region is sufficient to cause two-photon absorption by the two-photon absorption moiety; and
a detector configured to detect emissions generated at the reaction site resulting from two-photon absorption of the excitation energy by the two-photon absorption moiety.

2. The system of claim 1, further comprising a fluorescence resonant energy transfer (FRET)-based dye system reacted with the nucleic acid molecule at the reaction site.

3. The system of claim 2, wherein the two-photon absorption moiety is a donor of the FRET-based dye system.

4. The system of claim 3, wherein the detector is configured to detect emissions generated by fluorescence resonant energy transfer excitation of an acceptor of the FRET-based dye system reacted with the single molecule of nucleic acid at the reaction site.

5. The system of claim 4, wherein the acceptor is one of a plurality of differing organic dyes labeling each of four nucleotides to be incorporated by the single nucleic acid molecule.

6. The system of claim 2, wherein a donor of the FRET-based dye system is carried by a polymerase at the reaction site.

7. The system of claim 1, wherein the excitation energy is transmitted at a wavelength that is not sufficient for one-photon absorption by the two-photon absorption moiety.

8. The system of claim 1, wherein the excitation source comprises a laser source.

9. The system of claim 1, wherein the excitation source is chosen from a pulsed laser and a constant wavelength laser.

10. The system of claim 1, wherein the two-photon absorption moiety is chosen from at least one of a chromophore dye, a rhodamine dye, a quantum dot nanocrystal, a squaraine, a conjugated porphyrin, and an aromatic expanded porphyrin analogue.

11. The system of claim 1, wherein the two-photon absorption moiety is a dye labeling a nucleotide.

12. The system of claim 1, wherein the two-photon absorption moiety is one of a plurality of differing organic dyes labeling each of four nucleotides.

13. The system of claim 1, wherein the optical system comprises a beam splitter.

14. The system of claim 1, wherein the optical system comprises a confocal lens.

15. The system of claim 1, further comprising a detector filter configured to exclude passage of wavelengths of light including wavelengths longer than and within a wavelength range of the excitation and energy.

16. The system of claim 1, further comprising a flow cell within which the reaction site is disposed.

17. A method of detecting nucleic acids, the method comprising:
transmitting excitation energy to a reaction site at which a single molecule of nucleic acid is immobilized and reacted with a nucleotide labeled with a two-photon absorption dye;
focusing the transmitted excitation energy to a focal region containing the reaction site, wherein the excitation energy within the focal region is sufficient to cause two-photon absorption by the two-photon absorption dye;
detecting emissions generated at the reaction site by the two-photon absorption dye; and
determining a character or sequence of the single molecule of nucleic acid based on the detected emissions.

18. The method of claim 17, wherein determining the character or sequence of the single molecule of nucleic acid based on the detected emissions comprises sequencing the single molecule of nucleic acid.

19. The method of claim 17, wherein transmitting the excitation energy comprises transmitting the excitation energy at a wavelength that is not sufficient for one-photon absorption by the two-photon absorption dye.

20. The method of claim 17, wherein transmitting the excitation energy comprises transmitting the excitation energy to a reaction site at which the single molecule of nucleic acid is immobilized and reacted with a fluorescence resonance energy transfer (FRET)-based dye system.

21. The method of claim 20, wherein a donor of the FRET-based dye system is carried by a polymerase reacted with the single molecule of nucleic acid at the reaction site, and wherein said donor is a two-photon absorption moiety.

22. The method of claim 17, wherein transmitting the excitation energy comprises transmitting the excitation energy to a reaction site at which the single molecule of nucleic acid is immobilized and reacted with one of four nucleotides labeled with differing two-photon absorption dyes.

23. A method of detecting a nucleic acid molecule, comprising:
transmitting excitation energy to a reaction site at which a single molecule of nucleic acid is immobilized and reacted with a polymerase carrying a two-photon absorption moiety;
focusing the transmitted excitation energy to a focal region containing the reaction site, wherein the excitation energy within the focal region is sufficient to cause two-photon absorption by the two-photon absorption moiety;
detecting emissions from an acceptor dye at the reaction site which is excited from fluorescence resonance energy transfer (FRET) from the two-photon absorption moiety; and
determining a character or sequence of the nucleic acid molecule based on the detected emissions.
